(19) United States
(12) Patent Application Publication
(10) Pub. No.: US 2018/0185672 A1
(43) Pub. Date: Jul. 5, 2018

(54) DETERMINATION OF RADIATION COLLIMATOR COMPONENT POSITION

(71) Applicant: Sun Nuclear Corporation, Melbourne, FL (US)

(72) Inventor: Amir Ramezanzadeh Moghadam, Melbourne, FL (US)

(73) Assignee: Sun Nuclear Corporation

(21) Appl. No.: 15/395,852

(22) Filed: Dec. 30, 2016

(51) Int. Cl. A61N 5/10; G21K 1/04

(52) U.S. Cl. CPC A61N 5/1064; G21K 1/046; A61N 5/1077

(57) ABSTRACT

A system, a method, and a computer program product for determining a position of a collimator component of a radiation delivery device. Starting and ending times of an image generated by the radiation delivery device are synchronized with a radiation treatment plan executed by the radiation delivery device. The starting and ending times define a period of time when the generated image was acquired. Based on the synchronized starting and ending times, a predicted characteristic of the image is compared to a measured characteristic of the corresponding measured image. The predicted characteristic is determined by the radiation treatment plan. Based on the comparison, a position of the collimator component is determined. The determined position of the collimator component is compared to the radiation treatment plan and/or treatment log. The synchronized starting and ending times of the generated image are then adjusted.

### TECHNICAL FIELD

In some implementations, the current subject matter relates to radiation therapy systems, devices, and methods, and in particular, to a determination of an accurate position of one or more radiation collimator components.

### BACKGROUND

Radiation therapy is used to treat cancerous tumors with ionizing radiation that kills the affected cancer cells. External beam radiotherapy can be used to deliver ionizing radiation. In such therapy, a patient is placed on a couch and a radiotherapy beam generator is positioned to direct the ionizing radiation at the patient's tumor. A linear accelerator (“LINAC”) is typically used for the purposes of delivering external beam radiation treatments to patients. LINAC can deliver high-energy x-ray beam to the region of the target tissue, where the x-ray is sufficiently focused to destroy the target cells (e.g., tumor cells, abnormal cells, etc.), while avoiding the surrounding normal tissue.

One method for determining the proper positioning of the patient with respect to the beam is to use data from a radiation detector, for example, an electronic portal imaging device (EPID). Images from an EPID can be used to verify a dose received by the patient during a radiotherapy session. However, the detected collimator component(s) positions generated by conventional processes are highly inaccurate as they are not able to provide accurate positions of collimator component(s) in the presence of collimator component(s) motions or, in the alternative, require a high image acquisition frame rate to yield accurate results, thereby making such processes incompatible with existing radiotherapy systems.

### SUMMARY

In some implementations, the current subject matter relates to a computer implemented for determining position of a collimator component of a radiation delivery device. The method can include synchronizing a starting time and an ending time of an image generated by the radiation delivery device with a radiation treatment plan executed by the radiation delivery device. The starting and ending times define a period of time when the generated image was acquired. The method can further include comparing, based on the synchronized starting and ending times, at least one predicted characteristic of the image to at least one measured characteristic of a corresponding measured image. The predicted characteristic can be determined by the radiation treatment plan. Based on comparison, a position of the collimator component can be determined. The determined position of the collimator component can then be compared to the radiation treatment plan and/or treatment log. Based on the comparison, synchronized starting and ending times of the generated image can be adjusted.

In some implementations, the current subject matter can include one or more of the following optional features. The generated image can be generated by the radiation delivery device at a low frame rate, a low acquisition rate, and/or can include blurriness. In some exemplary, non-limiting implementations, the low frame rate can be less than 10 frames per second.

In some implementations, the predicted characteristic of the image can be determined based on at least one of a motion and a presence of the collimator component as defined in the radiation treatment plan. The measured characteristic of the image can be determined based on at least one of the measured image and a measured motion and a measured presence of the collimator component and a measured image.

In some implementations, the method can further include determining at least one of a motion and a presence of the collimator component based on the at least one measured characteristic of the measured and/or reconstructed image, and analyzing the measured characteristic and the predicted characteristic to determine a trustworthiness of the measured image. The determination can be based on at least one of the following: the radiation treatment plan, an anatomy of the patient (the patient can be identified in the radiation treatment plan), and/or at least another measured image acquired by the radiation delivery device in accordance with the radiation treatment plan.

In some implementations, the process can also include repeating, using the adjusted synchronized starting and ending times of the generated image, the comparing, the determining, and the adjusting operations.

In some implementations, the process can include refining the adjusted synchronized starting and ending times of the generated image, generating, based on the refined starting and ending times, a refined predicted characteristic (or a refined predicted profile of the collimator component), measuring, based on the generated refined predicted characteristic, a refined position of the collimator component, and adjusting, based on the measuring, the refined starting and ending times of the generated image.

In some implementations, comparison of the characteristics can include comparing a value of the measured characteristic of at least one point in the measured profile to a value of predicted corresponding characteristic of that point in the predicted profile. The characteristics can include at least one of the following: an amplitude, gradient, a slope, a derivative of a slope, an average of a plurality of amplitudes, a radius of curvature, an average of a plurality of slopes, and any combination and/or function of thereof. The measured image can be determined based on a measured profile of the collimator component. The predicted image can be determined based on a predicted profile of the collimator component as defined in the radiation treatment plan. Further, the measured profile of the collimator component can be determined using a centerline of the collimator component.

In some implementations, a trustworthiness of the determined position of the collimator component can be verified based on another position of the collimator component determined during at least a portion of the radiation treatment delivered by the radiation delivery device in accordance with the radiation treatment plan.

In some implementations, the radiation delivery device can include at least one of the following: an electronic portal imaging device, an array of radiation detectors, a diode array, a TFT array, an ionization chamber array, etc., and/or any combination thereof. Further, at least one of the synchronizing, the comparing, the determining, and the adjusting operations can be performed by at least one processor of at least one computing system. The computing system can include at least one of the following: a software component, a hardware component, and any combination thereof.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a computer-readable storage medium, may include, encode, store or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including but not limited to a connection over a network (e.g. the Internet, a wireless wide area network, a local area network, a wide area network, a wired network or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings and from the claims. While certain features of the currently disclosed subject matter are described for illustrative purposes, it should be readily understood that such features are not intended to be limiting. The claims that follow this disclosure are intended to define the scope of the protected subject matter.

### BRIEF DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 is a simplified diagram illustrating a radiation therapy system, according to some implementations of the current subject matter;

FIG. 2 illustrates an image acquired using a 10 frames per second acquisition rate;

FIG. 3 illustrates an image acquired at a 1 frame per second acquisition rate;

FIG. 4 illustrates an exemplary process for detecting collimator component(s) positions, according to some implementations of the current subject matter;

FIG. 5 illustrates an exemplary image that can be used for determination or extraction of measured profiles of MLC leaf pairs, according to some implementations of the current subject matter;

FIG. 6 illustrates an exemplary plot comparing predicted or expected profiles/positions to measured or detected profiles/positions, according to some implementations of the current subject matter;

FIG. 7 illustrates an exemplary process for refining starting and ending positions of an image, according to some implementations of the current subject matter;

FIG. 8 illustrates an exemplary system, according to some implementations of the current subject matter; and

FIG. 9 illustrates an exemplary process, according to some implementations of the current subject matter.

### DETAILED DESCRIPTION

In some implementations, the current subject matter relates to systems, methods, devices, and/or computer program products for determining an accurate position of a radiation collimator in a radiation delivery system. In some implementations, the current subject matter can perform an accurate verification of positions of collimator components regardless of presence of a blur in the images (e.g., low frequency images). The process can begin by synchronizing acquired images to a planned course of the treatment or treatment log(s). The planned course of treatment can typically be developed prior to the initiation of a treatment process and can define images that are expected to be acquired during 1-2 seconds during the image acquisition process. Then, a profile can be ascertained from the acquired images (i.e., electronic portal imaging devices (“EPID”) images) through a centerline of the multi-leaf collimator (“MLC”) leaf(s). Once the exact start and end of the image acquisition (i.e., starting and ending times) are determined, it is possible to predict the centerline profile of each MLC leaf pair. This prediction can be performed using an actual dose calculation engine, and/or a simplified physics model of the beam. This makes it possible to determine the expected shape of the MLC leaf(s) profile at positions where the MLC leaf(s) were expected to be. Using the expected shape of the profile at the extreme MLC leaf(s) positions, the corresponding measured profile extracted from the acquired low frame rate images can be searched to determine if there is a match. An iterative refining process can be performed to more accurately determine starting and ending times of each image as well as MLC leaf(s) positions. Additionally, the motion and/or the speed of the MLC leaf(s) during movement from one extreme position to another can be verified.

In some implementations, the current subject matter can perform one or more quality checks to verify if the type of the motion of the MLC leaf(s), the timing and the detected positions and/or motions were verifiable, trustable and/or not contradictory to each other. If contradictory measurements are detected, the measurement(s) that are closer to the remaining measurements can be kept and the other contradictory measurement(s) can be discarded and/or could be reprocessed using additional search constraints to determine new MLC leaf(s) position(s) that is/are not contradictory to other measurements.

Next, details of the events in the course of the treatment can be generated by combining information gathered from the images, logs and the treatment plan. The information from the images and logs can be compressed to reduce calculation time for the dose calculation engine and/or any other dose and/or fluence analysis tool, which can utilize the generated data.

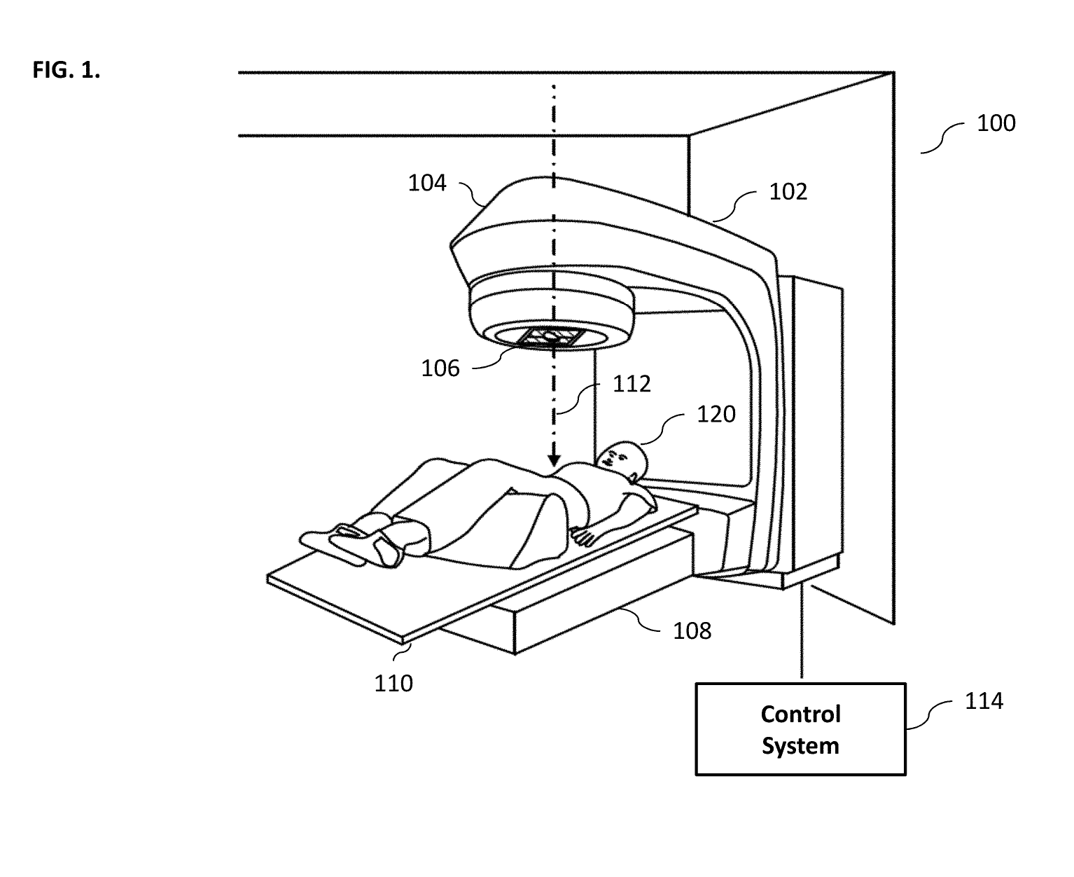

FIG. 1 illustrates an exemplary radiation system 100, according to some implementations of the current subject matter. The system 100 can include a radiation therapy device 102, a gantry 104, a beam collimator 106, a radiation detector (or a radiation detector array) 108, a patient couch 110, and a control system 114. During therapy, a patient 120 can be positioned on the patient couch 110 and a treatment beam 106 can be delivered to a target tissue of the patient. The target tissue can be previously identified through use of various scanning technologies. Patient's positioning on the patient couch 110 can also be predetermined so that the delivery of the beam is accurate. Operation of the system 100 can be controlled using the control system 114, which can include processors, computers, hardware, computer programs, and any combination thereof. The control system 114 can be used to determine how the beam 102 can be delivered to the patient, rotation of the gantry 104, positioning of the patient 112 as well as other parameters of the treatment process. The control system 114 can also be used to monitor patient 112 during the treatment (e.g., a treatment fraction) and/or change treatment parameters, if so required. The control system 114 can be used to perform quality assurance (“QA”) to ensure that accurate radiation treatment is delivered. As can be understood, the current subject matter is not limited to the setup shown in FIG. 1. The radiation system can be so designed so that it can deliver radiation therapy to any part of the human body, where only a portion of the human body can be placed in the vicinity of any component of the system.

In some implementations, the gantry 104 can rotate about the patient 120. The gantry 104 can include a radiation source such as a LINAC, cobalt 60 source, etc., that can direct radiation toward patient 120 by way of treatment beam 112. The treatment beam 112 can include scanning beams, where a small beamlet can be scanned over the area that is required to be treated. The treatment beam 112 can be shaped by the beam collimator 106 prior to application to the patient 120.

In some implementations, to deliver proper treatment to the patient, a radiation treatment plan can be developed. The plan can include, for example but not limited to, information about radiation delivery, delivery log information, time, dose information, treatment beam shape or energy, orientation of the gantry, collimator leaf positions, patient anatomy (CT) image orientation with respect to the treatment beam, any other measurements, and/or any other data.

The LINAC can be periodically tested to verify its ability to deliver radiation fields defined by a standardized machine QA protocol as well as its ability to deliver radiation fields defined by a treatment planning system (“TPS”) for a specific patient treatment. A variety of QA tools are available. These tools can include radiation sensitive detector arrays that include one, two and/or three dimensional (1D, 2D, 3D) spatial geometries (e.g., PROFILER™ (1D), IC PROFILER™ (1D, four orthogonal 1D arrays), MapCHECK® (2D), and ArcCHECK® (3D), as available from Sun Nuclear Corporation, Melbourne, Fla., USA).

The gantry angle parameter can be used to ensure accurate radiation delivery and can be tested as part of the radiation system QA. Test methods can include inclinometers that can be mounted to the gantry and an array system (e.g., ArcCHECK®, as available from Sun Nuclear Corporation, Melbourne, Fla., USA) that can analyze information from measurement of the beam passage through the 3D detector geometry. The gantry angle is also important for determining an accurate patient plan QA. This can be important for the purposes of gantry angle QA and for detector correction for dose measurements, when detectors' response to radiation includes an inherent response to beam direction. To improve radiation dose measurement during the patient QA at various beam angles, a correction to the measurement can be determined from the gantry angle and/or the directional response function. Determination of the gantry angle during radiation measurement can be indirect when determined using an inclinometer. This correction method can rely on performance of another sensor.

In some implementations, electronic portal imaging devices (“EPIDs”) can be used to verify radiation delivery to a patient. The EPIDs can provide images that can be used for verification of positioning of radiation collimating components, e.g., multi-leaf collimator (“MLC”) and jaws, which can be used to limit radiation field to prevent exposure of healthy tissue and only expose unhealthy (e.g., cancerous) tissue to radiation. Multi-leaf collimators constantly move throughout a treatment to generate complex shapes in order to optimize delivery of a maximum dose of radiation to tumorous tissue, while preserving healthy tissue in the patient. The leafs of the collimator are typically very heavy and have a complex range of motions. As such, to ensure proper delivery of radiation in accordance with the treatment plan, it is important to constantly verify positions of collimator leafs to prevent delivery errors (e.g., in case one of the components in the treatment machine fails to perform as expected).

There are two ways to verify dose delivered by the EPIDs. One of the methods is back projection algorithms and the other is forward projection algorithms. Back projection algorithms rely on a previous knowledge of the anatomy of the patient and can be sensitive to changes in the patient anatomy. Since patient anatomy changes every day through the course of the treatment, back projection algorithms can be susceptible to error.

Forward projection algorithms can use radiotherapy images in order to detect an actual position of different components of the radiation collimator (and hence, to verify delivery of radiation dose), and, as a result, are typically less susceptible to error that can be caused by patient anatomy changes because presence of collimator component(s) creates a much higher image contrast than the regular change in human anatomy. However, conventional forward projection algorithms usually require high frame rate image acquisition (e.g., 10 images per second) in order to be able to yield an accurate result. This high frame rate image acquisition is important to conventional forward projection algorithms because they require very minimal collimator component movement during image acquisition. This minimal collimator movement results in a high contrast image in which collimator components can be easily distinguished from patient anatomical attenuations using any conventional edge detection algorithm. However, this high frame rate image acquisition is typically not available on conventional EPIDs. Further, most conventional EPIDs have a computer memory limitation (usually, 256 images per delivery) that might not permit continuous image acquisition throughout radiation delivery of a single fraction (e.g., approximately two minutes) and which can require acquisition of 1200 images (e.g., 1.2 gigabytes of images for only one fraction). Hence, a full verification of the whole treatment can require storage of up to 60,000 images that may be part of a patient record and can be larger than 60 GB, thereby creating substantial network traffic and storage issues.

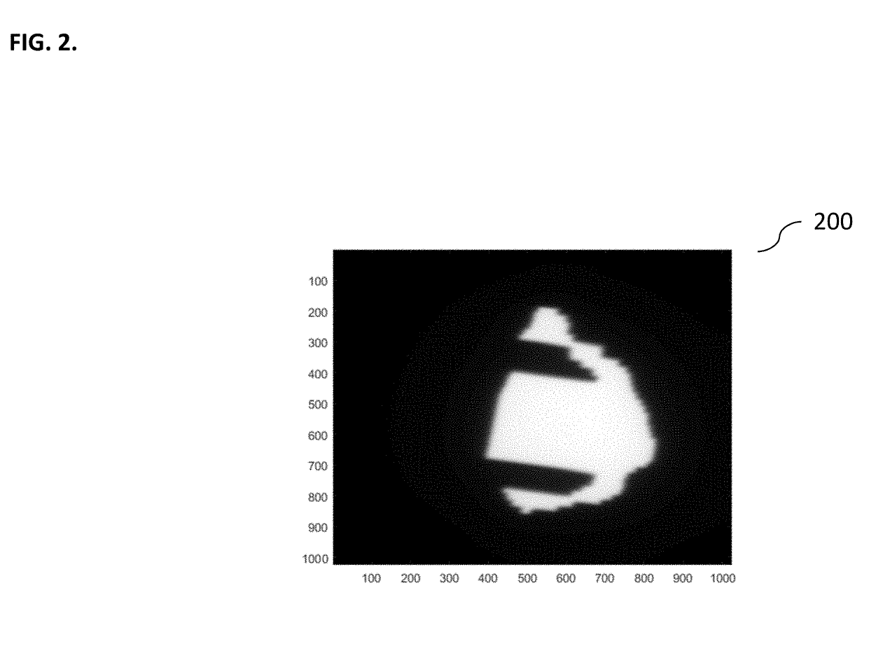

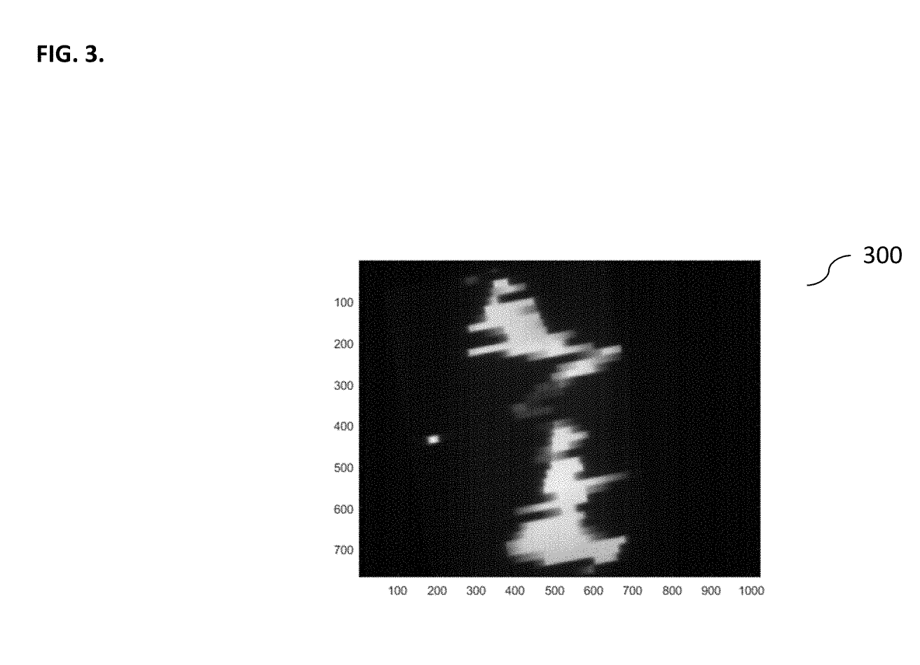

FIGS. 2-3 illustrate exemplary images that were acquired using conventional linear accelerator systems using different frame rates. FIG. 2 illustrates an image 200 acquired using a 10 frames per second acquisition rate. FIG. 3 illustrates an image 300 acquired at a 1 frame per second acquisition rate. As can be seen from FIG. 2, image 200 has sharp edges that can be easily detected using any conventional edge detection algorithm. As shown in FIG. 3, image 300 has blurry edges, where the blurriness is caused by the motion of the collimator components during the 1 second length of acquisition. In some cases, the collimator components can move as much as 2.5 cm during the 1 second image acquisition, thereby creating a 2.5 cm long blur in the image. Thus, the conventional edge detection algorithms typically fail to detect a correct position of the collimator components that were in motion during acquisition of the image.

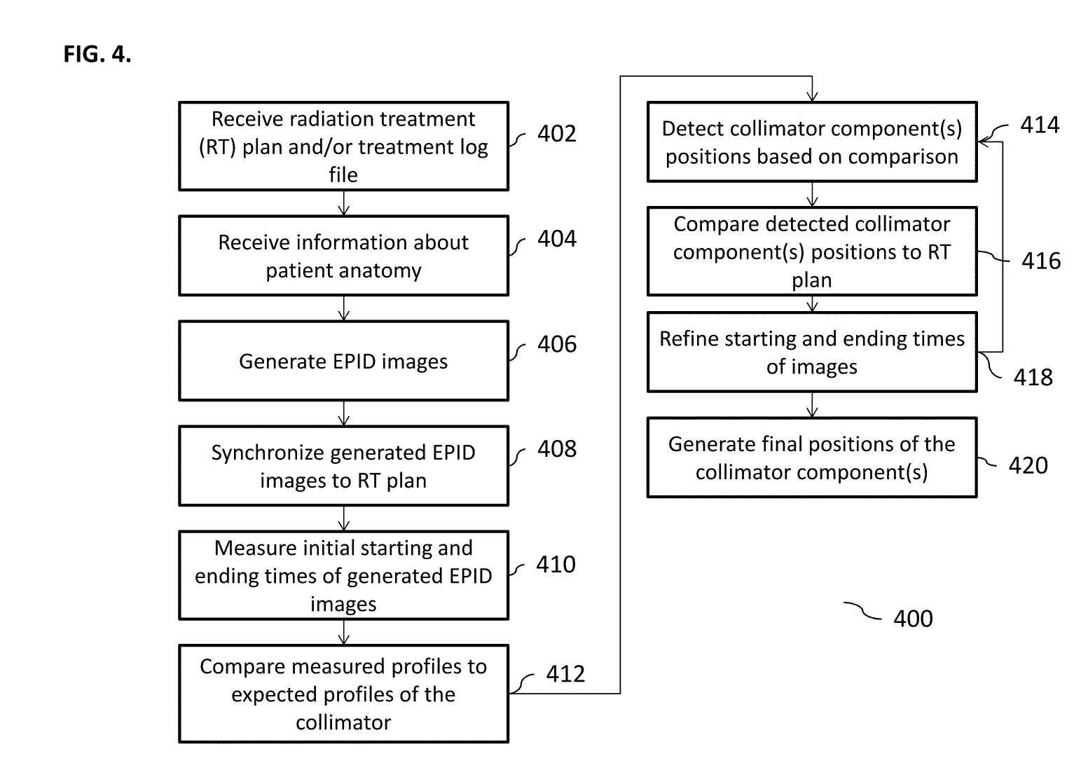

FIG. 4 illustrates an exemplary process 400 for detecting collimator component(s) (e.g., MLC leafs and/or jaws) positions, according to some implementations of the current subject matter. The process 400 can be performed by one or more components of the system 100 shown in FIG. 1. In some exemplary implementations, the process 400 can be performed by the control system 114 and/or any other computing components. The control system 114 and/or any other computing components can receive, transmit, and/or process various data, which can include any data, images, video, audio, text, programs, functions, etc. and/or any combination thereof. The data can be received/transmitted via a communications network, e.g., the Internet, an intranet, an extranet, a local area network (“LAN”), a wide area network (“WAN”), a metropolitan area network (“MAN”), a virtual local area network (“VLAN”), and/or any other network and/or any combination thereof. The data can be received/transmitted via a wireless, a wired, and/or any other type of connection. The processing of the data can be implemented using software, hardware and/or any combination of both. The processing can be performed using a personal computer, a laptop, a server, a mobile telephone, a smartphone, a tablet, and/or any other type of device and/or computing system and/or any combination thereof. The component(s) control system 114/computing components can be separate components and/or can be integrated into one or more single computing components.

Referring back to FIG. 4, the process 400 can determine accurate positions of the collimator components based on images acquired using low frame rates, which can be lower than 10 frames per second, which can be blurry. The process 400 can also utilize the knowledge of patient anatomy and planned collimator component(s) motions (as obtained from a treatment plan) along with obtained low frame rate images to detect collimator component(s) positions accurately. For ease of discussion only, collimator component(s) will be referred to as MLC leafs or MLC leaf pairs. As can be understood, the current subject matter processes can be applicable to any collimator component(s).

At 402, a radiation treatment (“RT”) plan or a treatment log file can be received by the system 100. At 404, patient information, including patient's CT scan, cone beam CT, Portal Images, Digitally reconstructed Radiographs (DRR), Mill and/or any other image(s) and/or information concerning patient's anatomy can be also provided to the system 100. At 406, the EPID images of a patient's fraction can be generated. The information obtained as a result of 402-406 can be part of a routine patient's radiation therapy treatment process/procedure.

At 408, the generated EPID images can be compared to and synchronized with the information (including any images) that is contained in the RT plan and/or treatment log(s). An exemplary synchronization process is disclosed in co-owned, co-pending U.S. patent application Ser. No. 14/694,865 to Moghadam et al., filed Apr. 23, 2015, and entitled “Radiation Detector Calibration”, the disclosure of which is incorporated herein by reference in its entirety. At 410, initial starting and ending times of the generated EPID images can be measured. Based on the starting and ending times of the generated EPID images, predicted MLC leaf(s) profiles (as can be determined utilizing the information in the RT plan/treatment log file and/or the anatomical images of the patient) can be compared to the measured MLC leaf(s) profiles, at 412. Alternatively and/or in addition to, predicted characteristic(s) of predicted images (as defined in the RT plan/treatment log and/or the anatomical image of the patient) can be compared to the measured characteristic(s) of the respective measured images. The characteristics can include at least one of the following: an amplitude, a slope, a gradient, a derivative of a slope, an average of a plurality of amplitudes, an average of a plurality of slopes, a radius of curvature, Longest Common Subsequence (LCSS), Dynamic Time Warping (DTW), Fréchet distance, Procrustes analysis, and/or any combination and/or function of thereof. The comparison can be used to detect collimator component(s) (e.g., MLC leafs and/or Jaws) positions, at 414. The detected collimator component(s) positions can be compared to the expected positions of the collimator component(s) as per RT plan/treatment log file, at 416. Using the comparison, the starting and ending times of the images can be refined, at 418. The operations 414-418 can be repeated to generate more refined starting and ending times of the images. In some implementations, the operations 414-418 can be repeated multiple times (e.g., five times and/or until the refining iteration results do not change as compared to a previous iteration). Once the process is complete, final positions of the collimator component(s) can be generated, at 420, and outputted.

In some implementations, to perform synchronization (at 408), the RT plan/treatment log file can be analyzed to determine fluence at intervals between control points along the path of each MLC leaf pair. By analyzing the RT plan/treatment log file, an expected track of one or more MLC leaf shadows can be determined. For each MLC leaf, a measured dose profile along the MLC centerline can be determined by extracting the measured signals corresponding to the EPID image pixels located along the profile. In some exemplary implementations, instead of using a single image pixel to represent a value on each point in the profile, an average of the adjacent pixels on the EPID, and/or all adjacent profiles in parallel can be used. A profile can be defined as an array of values extracted from images along a specific line (e.g., an imaginary line drawn along the center of the MLC leaf(s) in the direction of the MLC leaf(s) motion).

Further, gantry angle information contained in a DICOM header of each cinefluougraphy movie (“CINE”) frame can be used to estimate a starting time and an ending time of the image acquisition. Alternatively and/or in addition to, any other information contained in the DICOM header can be used to provide an approximation of duration of the image acquisition, and hence, a starting time and an ending time of the image. The approximated starting and ending times of the image can define an estimated acquisition time region.

Inside the estimated acquisition time region, an optimization search can be performed to determine exact starting and ending times of the image acquisition. The optimization search can result in different values for starting and ending times as compared to the initially determined values for the starting and ending times within the above region. A difference between the respective resulting times and initially determined times can be ascertained. The resulting times corresponding to the minimum difference in profile shapes from the predicted profiles can be selected as the actual starting and ending times of the image. In some implementations, the determined difference can be used as a measure of synchronization reliability. If the difference is larger than 50%, the process 400 can perform its optimization search in a wider region. If an acceptable synchronization is not found, an error can be generated. This can be indicative of an incorrect radiation delivery, error in the equipment, and/or any other problem. This can also be indicative that the MLC leaf(s) positions that will be subsequently determined can be subject to error(s) and can be disregarded if the remaining images imply that no delivery error(s) are present.

The optimization search can be performed for all EPID images that have been received, and as a result, most probable starting and ending times for all images can be determined. Based on the exact starting and ending times of each frame acquisition and expected (as per RT plan/treatment file log) MLC leaf(s) movements between these two times, predicted MLC leaf(s) profiles during these times can be determined. The predicted MLC leaf(s) profiles can be generated using any dose calculation engine, any physical and/or mathematical model, any analytical and/or artificial intelligence method, and/or any combination thereof. The predicted profiles can also include effects of attenuation, scattered radiation from the patient body that may be present in the radiation fields, noise, etc. For this purpose, the CT images of the patient (or any other imaging system that can provide information about the patient anatomy (such as, port images and Digitally reconstructed Radiographs (DRR)) can be used to predict effects of attenuation, scattered radiation from the patient body that may be present in the radiation fields, noise, etc. on the predicted MLC leaf(s) profile.

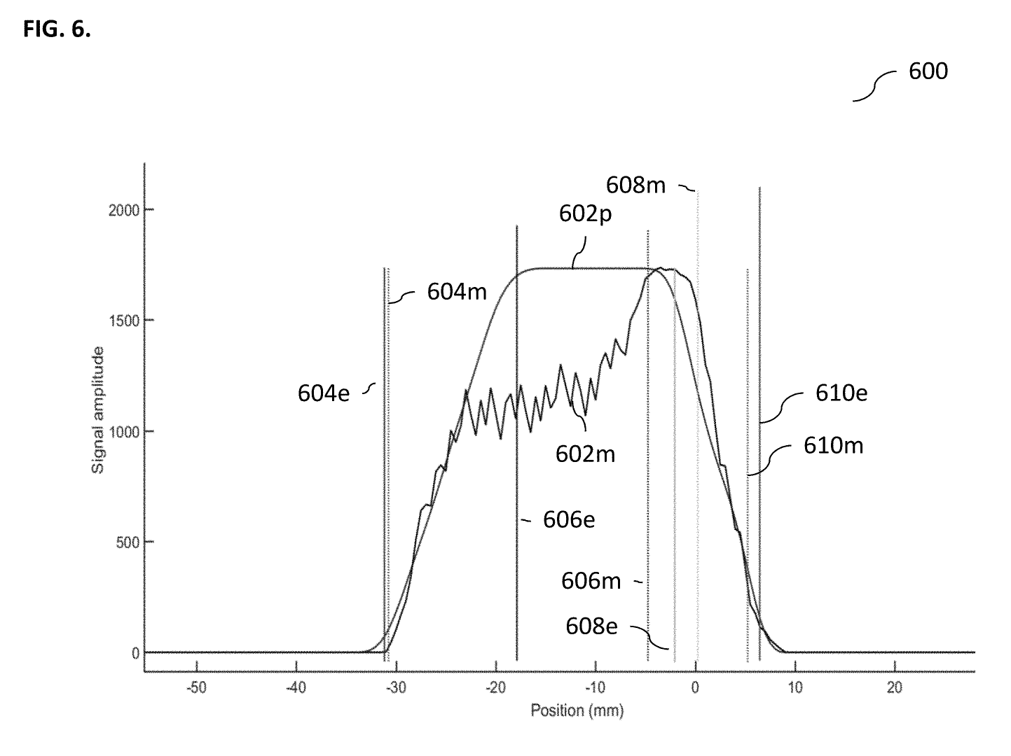

FIG. 6 illustrates an exemplary plot 600 comparing predicted or expected profiles/positions to measured or detected profiles/positions, according to some implementations of the current subject matter (in accordance with a conducted experiment). As shown in FIG. 6, a curve 602p (“p”—predicted) corresponds to a predicted profile of a pair of MLC leafs on a single 1 frame per second image and can be ascertained from RT plan/treatment file log and/or anatomical information from the patient. A curve 602m (“m”—measured) corresponds to a measured profile of MLC leaf(s). Vertical lines 604e (“e”—expected), 606e, 608e, and 610e correspond to expected positions 1, 2, 3, 4 of MLC leaf(s) along with respective expected signal amplitudes in accordance with RT plan/treatment file log. Vertical lines 604m, 606m, 608m, and 610m correspond to measured positions 1, 2, 3, 4 of MLC leaf(s) along with respective measured signal amplitudes. As shown in plot 600, while some expect and measured positions can be considered to be sufficiently close to one another (e.g., 604e and 604m), positions 606e and 606m are further apart from one another can be representative of an error. The error can be indicative of a radiation dose delivery error and/or any other errors. The plot 600 can be generated for each pair of MLC leaf(s) in accordance with the RT plan/treatment log file and can be used for determination of errors.

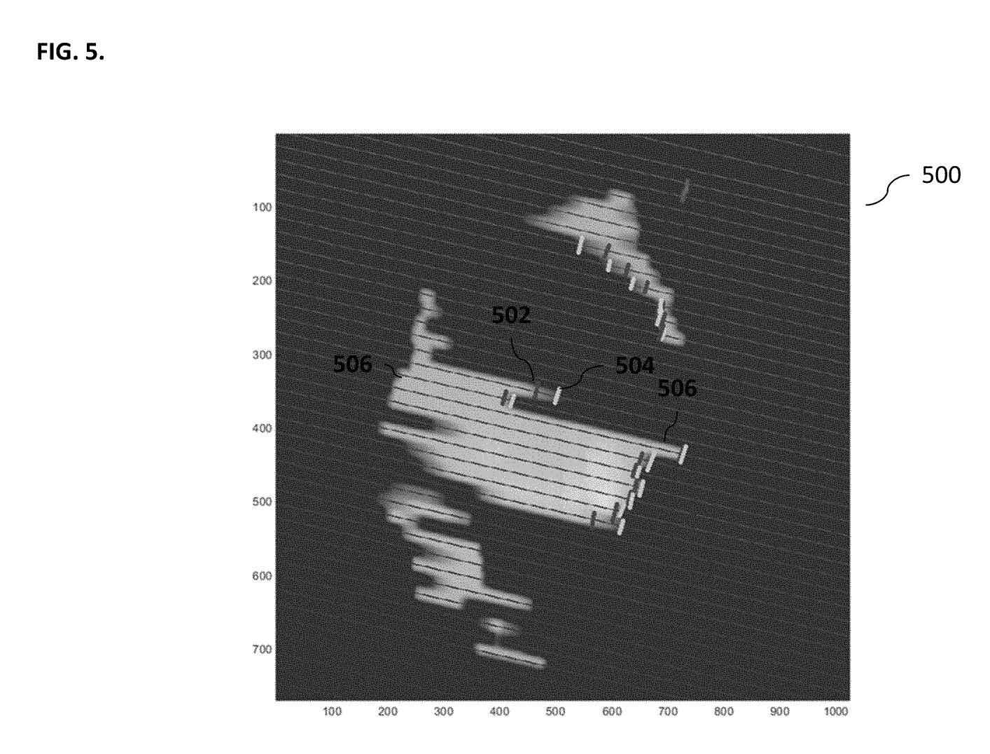

Referring back to FIG. 4, as stated above, once synchronization process is completed, the process 400 can perform measurement of or, otherwise, extract measured profiles for each MLC leaf pair. FIG. 5 illustrates an exemplary image 500 that can be used for determination or extraction of measured profiles of MLC leaf pairs. The measured profiles can be determined along line 506, which can correspond to values of the image extracted along the MLC leaf(s) centerline. In some implementations, the whole image and/or an average of the profiles and/or different parts of the image and/or individual pixel values can be analyzed one by one and/or in groups for the purposes of determining measured profiles. In some implementations, marks 502 (e.g., starting time) and 504 (ending time) can be included on the plot 500 to indicate extreme positions of MLC leaf(s) during radiation delivery. In some implementations, the detected motion of the MLC leaf(s) can be simulated on the same frame in a movie representation of the results.

The measured profiles extracted from the EPID image for each individual MLC leaf pair can be compared (at 412) to the expected profiles for the same MLC leaf pairs, as per RT plan/treatment log file, to determine the position of the MLC leaf(s) at times or points in time based on the similarity of their characteristics. These characteristics can include, but are not limited to, amplitude, slope, derivative(s) of the slope, curvature radius, average(s) of the amplitudes and/or slopes of a group of points and/or any combination and/or functions of the combinations of these characteristics in any dimensions (e.g., 1D, 2D, 3D, etc.). The points on the measured profile that have the closest characteristics to those points on the predicted profile can be selected as the measured (or detected) actual positions of the MLC leaf pair(s) that can be achieved using mathematical and/or morphological methods or using artificial intelligence. These points can correspond to two most extreme positions of the MLC leaf pair(s). A gradient between the two extreme points can provide information about the type of motion that the MLC leaf pair(s) experienced between these points. Further, the slope of the gradient can be used to determine the speed of the MLC leaf pair(s) between these points. For example, a uniform slope can correspond to a constant speed MLC leaf(s) and/or jaw motion.

In some implementations, the above predicted characteristics can be ascertained from the RT plan/treatment log file (without generating a predicted profile) and the above measured characteristics can be ascertained from the image (without generating a measured profile). Further, the measured MLC leaf(s) profile can be de-blurred and/or inversely de-attenuated, deconvolved and/or normalized. Also, the slope and/or signal amplitude and/or derivatives of different orders of the signal's amplitude of the de-attenuated profile between the two extreme MLC leaf(s) positions can be used to determine an exact trajectory of the MLC leaf(s) motion. Existing artificial intelligence and/or other optimization algorithms can be used to determine the trajectory once the two extreme locations are ascertained and that section of the profile is de-blurred and/or de-attenuated. In some implementations, the analysis can be performed in the Fourier space and/or any other transformation of the images into a different domain.

The values of the detected MLC leaf(s) positions (at 414) can be preliminary and can be further refined to more accurately determine positions of the MLC leaf(s) so that a proper QA analysis can be performed. These preliminary MLC leaf(s) positions can be iteratively compared to those indicated in the RT plan/treatment log file (at 416) to generate refined starting and ending times of the image(s) (at 418). In some implementations, the measured MLC positions at the beginning and the end of the image acquisition can be compared to the expected MLC leaf(s) positions at points of time that are close to the initial determination of the starting and ending time of the image acquisition, in order to minimize the difference caused by synchronization uncertainty. In alternate implementations, position and/or speed of the MLC leaf(s), either individually and/or in combination, can be used to optimize and/or minimize synchronization error(s). The refined starting and ending times can be used to generate a new set of predicted profile values, which in turn, can be used to measure a refined MLC leaf(s) positions for the same frame. This process of using the measured MLC leaf(s) positions for finding refined starting and ending times and using refined times for finding a new set of measured MLC leaf(s) positions can be repeated until there is a minimal change in values as compared to the previous set of values in the iteration.

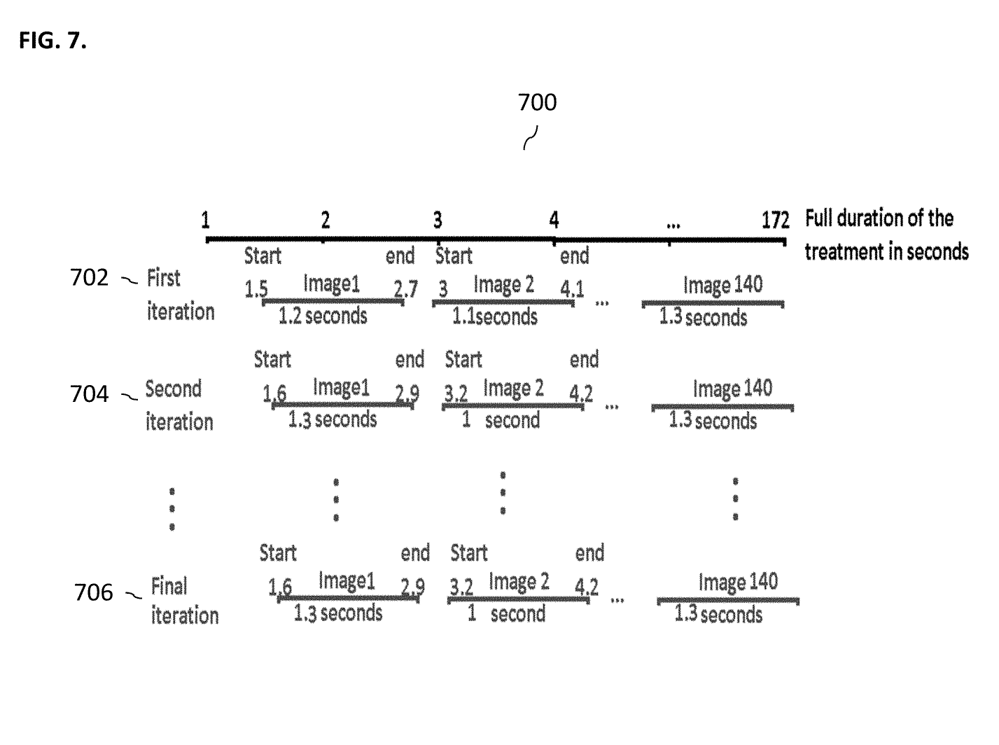

FIG. 7 illustrates an exemplary process 700 for refining starting and ending positions of an image, according to some implementations of the current subject matter. The process 700 can begin a first or initial determination 702 that resulted in starting and ending times for 140 images acquired during a treatment time of 172 seconds. For example, “Image1” was begun to be acquired at 1.5 second starting time and its acquisition ended 1.2 seconds later at 2.7 seconds. Similarly, acquisition of “Image2” started at 3.0 seconds and ended 1.1 seconds later at 4.1 seconds. Data for other images was likewise acquired.

Upon application of process 400 shown in FIG. 4, a second iteration 704 resulted in refined values for starting and ending times for the 140 images acquired during the initial determination 702. As shown in FIG. 7, the starting and ending times for the first refined “Image1” were adjusted to 1.6 seconds and 2.9 seconds, respectively, with an overall adjusted duration of 1.3 seconds; the starting and ending times for the second refined “Image2” were adjusted to 3.2 second and 4.2 seconds, respectively, with an overall adjusted duration of 1.0 second; etc. In the final iteration 706, the values associated with “Image1”, “Image2” and “Image140” remained unchanged. Hence, the values resulting from the final iteration 706 can serve as the values of the actual starting and ending times of the images and thus, the actual positions of the MLC leaf(s) can be determined (at 420 as shown in FIG. 4) for the purposes of QA analysis.

Radiation measurement devices typically suffer from various amounts of noise that can affect the results of the above algorithm, e.g., the result of the algorithm can indicate that positions of MLC leaf(s) are detected where MLC leaf(s) were not present. In some implementations, the current subject matter can perform a check of the final set of starting and ending times and MLC leaf(s) positions. As part of the check, the measured MLC leaf(s) positions can be inspected for contradictory values. If a contradictory set of measurements is found, the closest data points to the contradictory values can guide the algorithm towards finding the parts of the measured MLC positions that can be incorrect due to the presence of the noise in the data. If compressed images are used by the algorithm, where a significant amount of artifacts can make some of the images unusable, a secondary algorithm can inspect contrast and/or other expected properties in the image, such as the slope, rise, fall, noise level, histogram shapes, window and level values for the frame and/or any other properties of the image to determine affected frames and mark them for exclusion from the accurate MLC leaf(s) position detection analysis. In some cases, these images can be used for an overall verification of a small section of the radiation delivery and can be used to trigger an alert in the event they are extremely different from what was expected. The algorithm can use the knowledge of the treatment plan and/or anatomical images of the patient to determine if presence of high density patient organs (e.g., bones), low density organs (e.g., lungs), etc. can affect determination of correct positioning of the MLC leaf(s). If the radiation that passes adjacent to the MLC leaf(s) is planned to go through high density and/or low density organs, the algorithm can exclude that MLC leaf(s) position measurement in case it is contradictory with other nearby measurements for that MLC leaf(s), which are more trustable. The algorithm can also disregard the MLC leaf(s) motion measured by the analysis of the profile slope when the measured motion is extremely contradictory to the planned motion. The current subject matter can also utilize the MLC leaf(s) positions from the other beams of a patient treatment delivery to verify consistency of the positioning of MLC leaf(s) during a complete course of radiation delivery.

In some implementations, the measured MLC leaf(s) positions and/or the speed of the transitions along with the information from the RT plan/treatment log file can be used to generate a full measured record during treatment for verifying the radiation delivery. Further, for some sections of the delivery, there are no available EPID-based measured MLC leaf(s) positions. In this case, the current subject matter can use the shape of the motion of the MLC leaf(s) from the RT plan/treatment log file. The shape can be adjusted to fit to the two closest EPID measurement points on either side of such sections. For example, if there is no EPID measurement for a half of the treatment and particular MLC leaf(s) have always been off by 1 mm during the part of the delivery that had images available, the current subject matter can assume that the MLC leaf(s) position should be 1 mm off from the treatment log throughout the rest of the treatment for which images were not available and can generate corresponding values as the part of the final delivery events report.

The current subject matter process has numerous advantages. For example, the process can detect positions of the MLC leaf(s) with an uncertainty of +/−1 mm for 98% of the actual MLC leaf(s) positions using a 1 frame per 1.2 second image acquisition rate. This is very close to the results of the methods utilizing 10 frames per second images. Further, the current subject matter overcomes the problems of conventional systems performing verification of accuracy of radiation received by the patient. In particular, most conventional systems are limited to 10 frame-per-second imaging, which is not practical and on most systems may be impossible. The current subject matter can perform image based verification on all machines that are equipped with any imaging system. Additionally, conventional systems perform verification of images at 10 discrete points of time during one second (if the imaging system is capable of acquiring 10 frames per second), however the current subject matter is capable of verifying the MLC leaf(s) position(s) as a continuous function of time during the delivery using both high and/or low frame rate images.

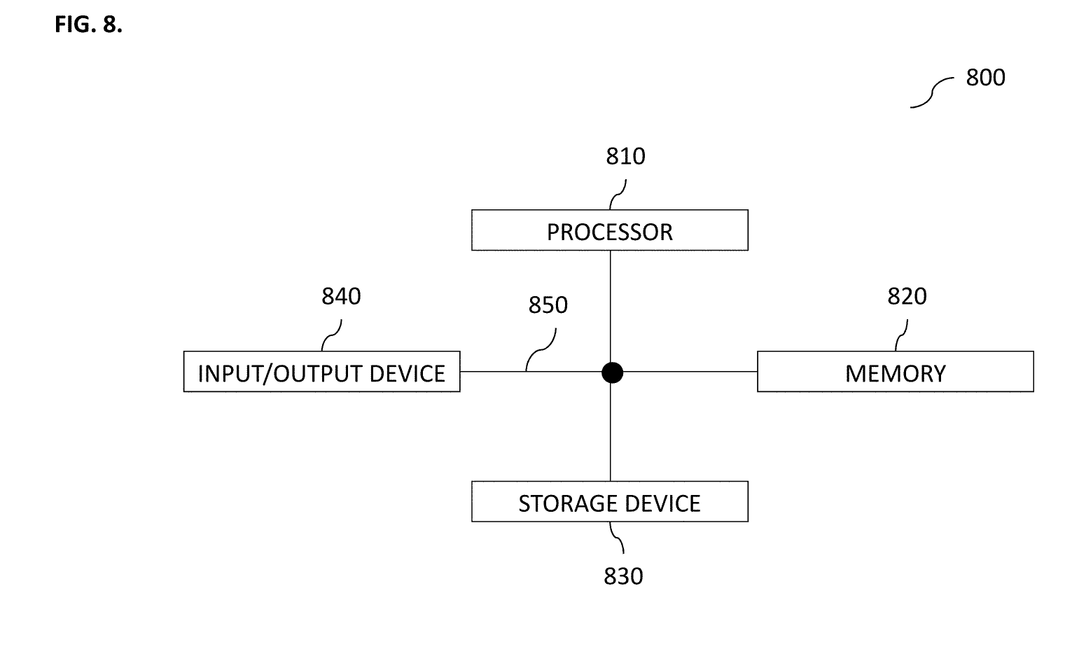

In some implementations, the current subject matter can be configured to be implemented in a system 800, as shown in FIG. 8. The system 800 can include one or more of a processor 810, a memory 820, a storage device 830, and an input/output device 840. Each of the components 810, 820, 830 and 840 can be interconnected using a system bus 850. The processor 810 can be configured to process instructions for execution within the system 400. In some implementations, the processor 810 can be a single-threaded processor. In alternate implementations, the processor 810 can be a multi-threaded processor. The processor 810 can be further configured to process instructions stored in the memory 820 or on the storage device 830, including receiving or sending information through the input/output device 840. The memory 820 can store information within the system 800. In some implementations, the memory 820 can be a computer-readable medium. In alternate implementations, the memory 820 can be a volatile memory unit. In yet some implementations, the memory 820 can be a non-volatile memory unit. The storage device 830 can be capable of providing mass storage for the system 800. In some implementations, the storage device 830 can be a computer-readable medium. In alternate implementations, the storage device 830 can be a floppy disk device, a hard disk device, an optical disk device, a tape device, non-volatile solid-state memory, or any other type of storage device. The input/output device 840 can be configured to provide input/output operations for the system 800. In some implementations, the input/output device 840 can include a keyboard and/or pointing device. In alternate implementations, the input/output device 840 can include a display unit for displaying graphical user interfaces.

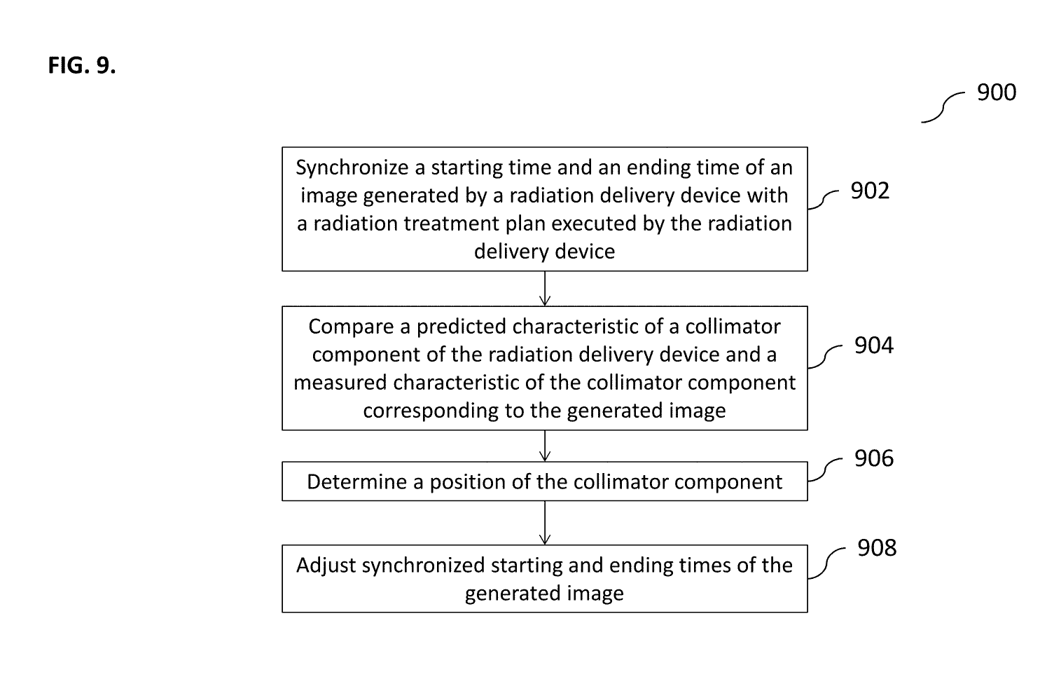

FIG. 9 illustrates an exemplary process 900 for determining collimator component position, according to some implementations of the current subject matter. At 902, a starting time and an ending time of an image generated by a radiation delivery device (e.g., radiation detector, etc.) can be synchronized with a radiation treatment plan executed by the radiation delivery device. The starting time and the ending time can define a period of time when the generated image was acquired. At 904, based on the synchronized starting and ending times, a predicted characteristic of an image (or alternatively a predicted profile of a collimator component (e.g., MLC leaf(s) and/or jaws) of the radiation delivery device) can be compared to a measured characteristic of a measured image (or alternatively a measured profile of the collimator component). The predicted characteristic can be determined by the radiation treatment plan. At 906, based on the comparison, a position of the collimator component can be determined. At 908, based on a comparison of the determined position of the collimator component to the radiation treatment plan and/or treatment log, synchronized starting and ending times of the generated image can be adjusted.

In some implementations, the current subject matter can include one or more of the following optional features. The generated image can be generated by the radiation delivery device at a low frame rate, a low acquisition rate, and/or can include blurriness. In some exemplary, non-limiting implementations, the low frame rate can be less than 10 frames per second.

In some implementations, the predicted characteristic of the image can be determined based on at least one of a motion and a presence of the collimator component as defined in the radiation treatment plan. The measured characteristic of the image can be determined based on at least one of the measured image and a measured motion and a measured presence of the collimator component and a measured image.

In some implementations, the method 900 can further include determining at least one of a motion and a presence of the collimator component based on the at least one measured characteristic of the measured and/or reconstructed image, and analyzing the measured characteristic and the predicted characteristic to determine a trustworthiness of the measured image. The determination can be based on at least one of the following: the radiation treatment plan, an anatomy of the patient (the patient can be identified in the radiation treatment plan), and/or at least another measured image acquired by the radiation delivery device in accordance with the radiation treatment plan.

In some implementations, the process 900 can further include repeating, using the adjusted synchronized starting and ending times of the generated image, the comparing, the determining, and the adjusting operations.

In some implementations, the process 900 can also include refining the adjusted synchronized starting and ending times of the generated image, generating, based on the refined starting and ending times, a refined predicted characteristic (or a refined predicted profile of the collimator component), measuring, based on the generated refined predicted characteristic, a refined position of the collimator component, and adjusting, based on the measuring, the refined starting and ending times of the generated image.

In some implementations, comparison of the characteristics can include comparing a value of the measured characteristic of at least one point in the measured profile to a value of predicted corresponding characteristic of that point in the predicted profile. The characteristics can include at least one of the following: an amplitude, gradient, a slope, a derivative of a slope, an average of a plurality of amplitudes, a radius of curvature, an average of a plurality of slopes, and any combination and/or function of thereof. The measured image can be determined based on a measured profile of the collimator component. The predicted image can be determined based on a predicted profile of the collimator component as defined in the radiation treatment plan. Further, the measured profile of the collimator component can be determined using a centerline of the collimator component.

In some implementations, a trustworthiness of the determined position of the collimator component can be verified based on another position of the collimator component determined during at least a portion of the radiation treatment delivered by the radiation delivery device in accordance with the radiation treatment plan.

In some implementations, the radiation delivery device can include at least one of the following: an electronic portal imaging device, an array of radiation detectors, a diode array, a TFT array, an ionization chamber array, etc., and/or any combination thereof. Further, at least one of the synchronizing, the comparing, the determining, and the adjusting operations can be performed by at least one processor of at least one computing system. The computing system can include at least one of the following: a software component, a hardware component, and any combination thereof.

One or more aspects or features of the subject matter described herein can be realized in digital electronic circuitry, integrated circuitry, specially designed application specific integrated circuits (ASICs), field programmable gate arrays (FPGAs) computer hardware, artificial neural networks, firmware, software and/or combinations thereof. These various aspects or features can include implementation in one or more computer programs that are executable and/or interpretable on a programmable system including at least one programmable processor, which can be special or general purpose, coupled to receive data and instructions from and to transmit data and instructions to, a storage system, at least one input device and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to programs, software, software applications, applications, components or code, include machine instructions for a programmable processor and can be implemented in a high-level procedural language, an object-oriented programming language, a functional programming language, a logical programming language and/or in assembly/machine language. As used herein, the term “machine-readable medium” refers to any computer program product, apparatus and/or device, such as for example magnetic discs, optical disks, memory and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback or tactile feedback; and input from the user may be received in any form, including, but not limited to, acoustic, speech or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone or A and B together.” A similar interpretation is also intended for lists including three or more items. For example, the phrases “at least one of A, B and C;” “one or more of A, B and C;” and “A, B and/or C” are each intended to mean “A alone, B alone, C alone, A and B together, A and C together, B and C together or A and B and C together.” Use of the term “based on,” above and in the claims is intended to mean, “based at least in part on,” such that an unrecited feature or element is also permissible.

The subject matter described herein can be embodied in systems, apparatus, methods and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown or sequential order, to achieve desirable results. Other implementations may be within the scope of the following claims.

What is claimed is:

1. A computer-implemented comprising:
synchronizing a starting time and an ending time of an image generated by a radiation delivery device with a radiation treatment plan executed by the radiation delivery device, wherein the starting time and the ending time define a period of time when the generated image was acquired;
comparing, based on the synchronized starting and ending times, at least one predicted characteristic of the image to at least one measured characteristic of a corresponding measured image, the at least one predicted characteristic being determined by the radiation treatment plan;
determining, based on the comparing, a position of the collimator component; and
adjusting, based on a comparison of the determined position of the collimator component to the radiation treatment plan and/or treatment log, synchronized starting and ending times of the generated image.

2. The method according to claim 1, wherein the generated image is generated by the radiation delivery device using at least one of: a low frame rate and a low acquisition rate.

3. The method according to claim 1, wherein
the at least one predicted characteristic of the image is determined based on at least one of a motion and a presence of the collimator component and a parameter of radiation as defined in the radiation treatment plan; and
the at least one measured characteristic of the image is determined based on at least one of the measured image and a measured motion and a measured presence of the collimator component.

4. The method according to claim 1, further comprising
determining at least one of a motion and a presence of the collimator component based on the at least one measured characteristic of the measured image; and
analyzing the at least one measured characteristic and the at least one predicted characteristic to determine a trustworthiness of the measured image, wherein the determination is based on at least one of the following: the radiation treatment plan, an anatomy of the patient, and/or at least another measured image acquired by the radiation delivery device in accordance with the radiation treatment plan.

5. The method according to claim 1, further comprising
repeating, using the adjusted synchronized starting and ending times of the generated image, the comparing, the determining, and the adjusting.

6. The method according to claim 1, further comprising
refining the adjusted synchronized starting and ending times of the generated image;
generating, based on the refined starting and ending times, a refined predicted characteristic;
measuring, based on the generated refined predicted characteristic, a refined position of the collimator component; and
adjusting, based on the measuring, the refined starting and ending times of the generated image.

7. The method according to claim 1, wherein the comparing further comprises
comparing a value of the at least one measured characteristic of at least one point in the measured image to a value of the at least one corresponding predicted characteristic of the at least one point in the predicted image;
wherein the measured and predicted characteristic include at least one of the following: an amplitude, a slope, gradient, a derivative of a slope, an average of a plurality of amplitudes, an average of a plurality of slopes, a radius of curvature, and any combination or function of thereof;
wherein the measured image is determined based on a measured profile of the collimator component, and the predicted image is determined based on a predicted profile of the collimator component as defined in the radiation treatment plan.

8. The method according to claim 7, wherein the measured profile of the collimator component is determined using a centerline of the collimator component.

9. The method according to claim 1, wherein a trustworthiness of the determined position of the collimator component is verified based on another position of the collimator component determined during at least a portion of the radiation treatment delivered by the radiation delivery device in accordance with the radiation treatment plan.

10. The method according to claim 1, wherein the radiation delivery device includes at least one of the following: an electronic portal imaging device, an array of radiation detectors, a diode array, a TFT arrays, an ionization chamber array, and any combination thereof.

11. The method according to claim 1, wherein at least one of the synchronizing, the comparing, the determining, and the adjusting is performed by at least one processor of at least one computing system, and wherein the computing system comprises at least one of the following: a software component, a hardware component, and any combination thereof.

12. A system comprising:
at least one programmable processor; and
a machine-readable medium storing instructions that, when executed by the at least one programmable processor, cause the at least one programmable processor to perform operations comprising:
synchronizing a starting time and an ending time of an image generated by a radiation delivery device with a radiation treatment plan executed by the radiation delivery device, wherein the starting time and the ending time define a period of time when the generated image was acquired;
comparing, based on the synchronized starting and ending times, at least one predicted characteristic of the image to at least one measured characteristic of a corresponding measured image, the at least one predicted characteristic being determined by the radiation treatment plan;
determining, based on the comparing, a position of the collimator component; and
adjusting, based on a comparison of the determined position of the collimator component to the radiation treatment plan and/or treatment log, synchronized starting and ending times of the generated image.

13. The system according to claim 12, wherein the generated image is generated by the radiation delivery device using at least one of: a low frame rate and a low acquisition rate.

14. The system according to claim 12, wherein
the at least one predicted characteristic of the image is determined based on at least one of a motion and a presence of the collimator component and a parameter of radiation as defined in the radiation treatment plan; and
the at least one measured characteristic of the image is determined based on at least one of the measured image and a measured motion and a measured presence of the collimator component.

15. The system according to claim 12, wherein the operations further comprise
determining at least one of a motion and a presence of the collimator component based on the at least one measured characteristic of the measured image; and
analyzing the at least one measured characteristic and the at least one predicted characteristic to determine a trustworthiness of the measured image, wherein the determination is based on at least one of the following: the radiation treatment plan, an anatomy of the patient, and/or at least another measured image acquired by the radiation delivery device in accordance with the radiation treatment plan.

16. The system according to claim 12, wherein the operations further comprise
repeating, using the adjusted synchronized starting and ending times of the generated image, the comparing, the determining, and the adjusting.

17. The system according to claim 12, wherein the operations further comprise
refining the adjusted synchronized starting and ending times of the generated image;
generating, based on the refined starting and ending times, a refined predicted characteristic;
measuring, based on the generated refined predicted characteristic, a refined position of the collimator component; and
adjusting, based on the measuring, the refined starting and ending times of the generated image.

18. The system according to claim 12, wherein the comparing further comprises
comparing a value of the at least one measured characteristic of at least one point in the measured image to a value of the at least one corresponding predicted characteristic of the at least one point in the predicted image;
wherein the measured and predicted characteristic include at least one of the following: an amplitude, a slope, gradient, a derivative of a slope, an average of a plurality of amplitudes, an average of a plurality of slopes, a radius of curvature, and any combination or function of thereof;
wherein the measured image is determined based on a measured profile of the collimator component, and the predicted image is determined based on a predicted profile of the collimator component as defined in the radiation treatment plan.

19. The system according to claim 18, wherein the measured profile of the collimator component is determined using a centerline of the collimator component.

20. The system according to claim 12, wherein a trustworthiness of the determined position of the collimator component is verified based on another position of the collimator component determined during at least a portion of the radiation treatment delivered by the radiation delivery device in accordance with the radiation treatment plan.

21. The system according to claim 12, wherein the radiation delivery device includes at least one of the following: an electronic portal imaging device, an array of radiation detectors, a diode array, a TFT arrays, an ionization chamber array, and any combination thereof.

22. A computer program product comprising a non-transitory machine-readable medium storing instructions that, when executed by at least one programmable processor, cause the at least one programmable processor to perform operations comprising:
synchronizing a starting time and an ending time of an image generated by a radiation delivery device with a radiation treatment plan executed by the radiation delivery device, wherein the starting time and the ending time define a period of time when the generated image was acquired;
comparing, based on the synchronized starting and ending times, at least one predicted characteristic of the image to at least one measured characteristic of a corresponding measured image, the at least one predicted characteristic being determined by the radiation treatment plan;
determining, based on the comparing, a position of the collimator component; and
adjusting, based on a comparison of the determined position of the collimator component to the radiation treatment plan and/or treatment log, synchronized starting and ending times of the generated image.

23. The computer program product according to claim 22, wherein the generated image is generated by the radiation delivery device using at least one of: a low frame rate and a low acquisition rate.

24. The computer program product according to claim 22, wherein
the at least one predicted characteristic of the image is determined based on at least one of a motion and a presence of the collimator component and a parameter of radiation as defined in the radiation treatment plan; and
the at least one measured characteristic of the image is determined based on at least one of the measured image and a measured motion and a measured presence of the collimator component.

25. The computer program product according to claim 22, wherein the operations further comprise
determining at least one of a motion and a presence of the collimator component based on the at least one measured characteristic of the measured image; and
analyzing the at least one measured characteristic and the at least one predicted characteristic to determine a trustworthiness of the measured image, wherein the determination is based on at least one of the following: the radiation treatment plan, an anatomy of the patient, and/or at least another measured image acquired by the radiation delivery device in accordance with the radiation treatment plan.

26. The computer program product according to claim 22, wherein the operations further comprise
repeating, using the adjusted synchronized starting and ending times of the generated image, the comparing, the determining, and the adjusting.

27. The computer program product according to claim 22, wherein the operations further comprise
refining the adjusted synchronized starting and ending times of the generated image;
generating, based on the refined starting and ending times, a refined predicted characteristic;
measuring, based on the generated refined predicted characteristic, a refined position of the collimator component; and
adjusting, based on the measuring, the refined starting and ending times of the generated image.

28. The computer program product according to claim 22, wherein the comparing further comprises
comparing a value of the at least one measured characteristic of at least one point in the measured image to a value of the at least one corresponding predicted characteristic of the at least one point in the predicted image;
wherein the measured and predicted characteristic include at least one of the following: an amplitude, a slope, gradient, a derivative of a slope, an average of a plurality of amplitudes, an average of a plurality of slopes, a radius of curvature, and any combination or function of thereof;
wherein the measured image is determined based on a measured profile of the collimator component, and the predicted image is determined based on a predicted profile of the collimator component as defined in the radiation treatment plan.

29. The computer program product according to claim 28, wherein the measured profile of the collimator component is determined using a centerline of the collimator component.

30. The computer program product according to claim 22, wherein a trustworthiness of the determined position of the collimator component is verified based on another position of the collimator component determined during at least a portion of the radiation treatment delivered by the radiation delivery device in accordance with the radiation treatment plan.

31. The computer program product according to claim 22, wherein the radiation delivery device includes at least one of the following: an electronic portal imaging device, an array of radiation detectors, a diode array, a TFT arrays, an ionization chamber array, and any combination thereof.
